(19) United States
(12) Patent Application Publication
(10) Pub. No.: US 2018/0256408 A1
(43) Pub. Date: Sep. 13, 2018

(54) METHOD AND APPARATUS FOR MAKING A NON-WOVEN FABRIC ARTICLE

(71) Applicants: Chen-Cheng HUANG, Taipei City (TW); Pao-Hao HUANG, Taipei City (TW); Pao-Han HUANG, Taipei City (TW)

(72) Inventors: Chen-Cheng HUANG, Taipei City (TW); Pao-Hao HUANG, Taipei City (TW); Pao-Han HUANG, Taipei City (TW)

(21) Appl. No.: 15/976,478

(22) Filed: May 10, 2018

Related U.S. Application Data
(63) Continuation-in-part of application No. 15/835,424, filed on Dec. 7, 2017.
Application No. 15/835,424 is a division of application No. 14/687,522, filed on Apr. 15, 2015, now Pat. No. 9,877,876.

(30) Foreign Application Priority Data
Apr. 18, 2014 (TW) 103114210

(51) Int. Cl. A61F 13/15; A61F 13/512; D04H 1/732; D04H 3/16; B29C 47/00

(52) U.S. Cl. CPC A61F 13/15203; A61F 13/5121; D04H 1/732; D04H 3/16; B29L 2031/4878; B29C 47/0028; B29C 47/003; B29C 47/0021

(57) ABSTRACT

A method and an apparatus for making a non-woven fabric article are provided. The method includes providing a screen mold, disposing a formable material thereover, and evacuating air from the screen mold. The screen mold is made of a non-pliable material, and has an annular surrounding wall that defines an internal space, and multiple screen holes that extend through the annular surrounding wall and communicate with the internal space. The formable material is disposed over the annular surrounding wall and the screen holes of the screen mold, and then air is evacuated from the internal space via a suction device to form the fabric article.

### CROSS-REFERENCE TO RELATED APPLICATION

This application is a continuation-in-part of U.S. patent application Ser. No. 15/835,424 (filed on Dec. 7, 2017), which is a divisional application of U.S. patent application Ser. No. 14/687,522 (filed on Apr. 15, 2015) that claims priority of Taiwanese Patent Application No. 103114210 (filed on Apr. 18, 2014). This application claims the benefits and priority of all these prior applications and incorporates by reference the contents of these prior applications in their entirety.

### FIELD

The disclosure relates to a method and an apparatus for making a non-woven fabric article, and more particularly to a method and an apparatus for making a breathable non-woven fabric article.

### BACKGROUND

Most garments nowadays are made from a conventional woven fabric article, in which gaps are generated by knitting vertically and horizontally. Such garments have good breathability and easy-drying properties. On the other hand, a conventional non-woven fabric article is manufactured by extrusion-molding a polymer melt feed into a plurality of semi-molten fibers by a melt-blowing process. The thus-formed semi-molten fibers are then stacked on one another, forming a thin layer of the non-woven fabric article.

Because the aforesaid non-woven fabric article is composed of multiple stacked fibers, garments made of such article consist of many small pores and hence have certain breathability and easy-drying properties. However, the breathability and easy-drying properties of the aforesaid non-woven fabric article still have limitations, e.g., if the density of the stacked molten-fibers is reduced in order to increase the ventilation and easy-drying effects, certain properties of the non-woven fabric article will be affected, such as elasticity, ductility, and others. Therefore, improvements in making a non-woven fabric article are needed in order to not only enhance the breathability and easy-drying properties, but also to maintain the original merits.

### SUMMARY

Therefore, an object of the disclosure is to provide a method and an apparatus for making a non-woven fabric article, which can alleviate at least one of the drawbacks of the prior art.

According to one aspect of the disclosure, a method of making a non-woven fabric article includes:
a) providing a screen mold made of a non-pliable material, and having an annular surrounding wall that defines an internal space, and a plurality of screen holes that extend through the annular surrounding wall and spatially communicate with the internal space;
b) disposing a formable material over the annular surrounding wall and the screen holes of the screen mold; and
c) evacuating air from the internal space via a suction device to form the formable material into the fabric article.

According to another aspect of the disclosure, an apparatus for making a non-woven fabric article includes a screen mold and a suction device. The screen mold is made of a non-pliable material, and has an annular surrounding wall that defines an internal space, and a plurality of screen holes that extend through the annular surrounding wall and spatially communicate with the internal space. The suction device is connected to the screen mold and is operable to evacuate air from the internal space of the screen mold.

According to yet another aspect of the disclosure, a fabric article including a non-woven wearable unit having a seamless non-woven sleeve is provided.

### BRIEF DESCRIPTION OF THE DRAWINGS

Other features and advantages of the present disclosure will become apparent in the following detailed description of the embodiment with reference to the accompanying drawings, of which:

FIG. 1 is a flow chart illustrating an embodiment of a method for making a non-woven fabric article according to the present disclosure;

FIG. 2 is a schematic view illustrating an embodiment of an apparatus for making a non-woven fabric article according to the present disclosure;

FIG. 3 is a fragmentary, partially-sectional perspective view illustrating a configuration of a screen mold of the apparatus;

FIG. 4 is a fragmentary sectional view illustrating that the screen mold has an internal space;

FIG. 5 is a schematic view illustrating that the screen mold is draped with a melt-blown fiber layer formed from molten fibers; and

FIG. 6 is a schematic view illustrating evacuation of air from the screen mold via a suction device.

### DETAILED DESCRIPTION

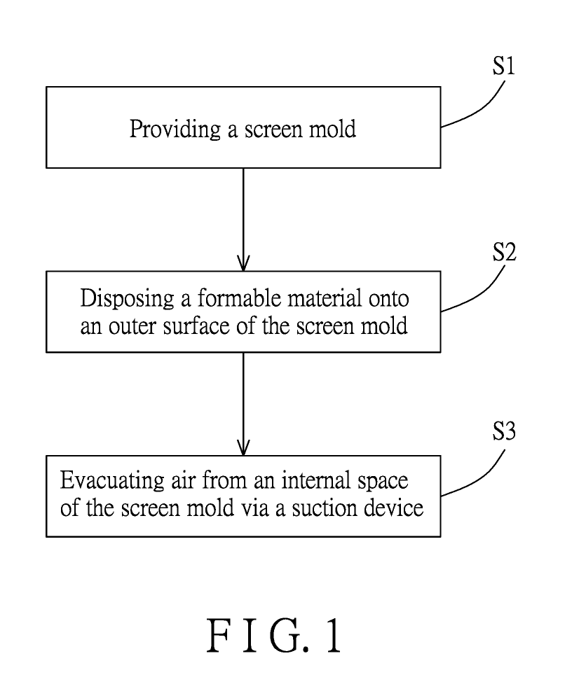

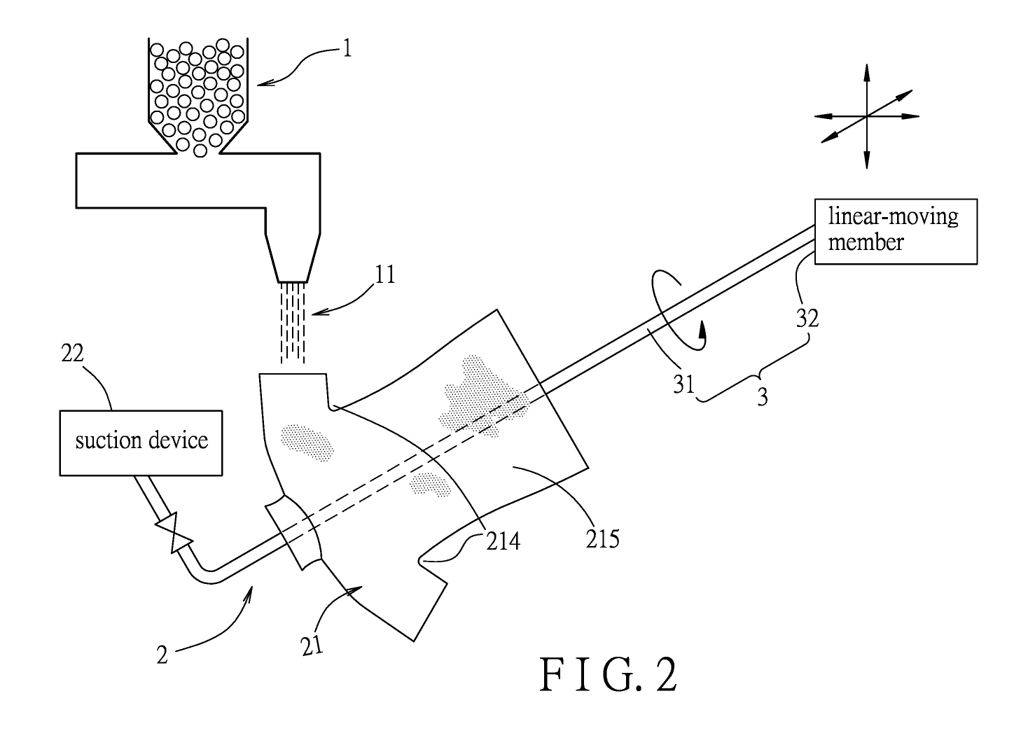

Referring to FIGS. 1 and 2, an embodiment of a method for making a non-woven fabric article according to the present disclosure, as well as an embodiment of an apparatus for the same purpose according to the present disclosure, is illustrated. For the sake of brevity, the method and the apparatus are described together below.

The method involves a melt-blown process and includes three steps (S1, S2 and S3). Generally speaking, molten fibers 11 produced from a melt-blown device 1 are sprayed onto an apparatus 2 for making a non-woven fabric article. The apparatus 2 includes a screen mold 21 that is hollow and a suction device 22 connected to the screen mold 21.

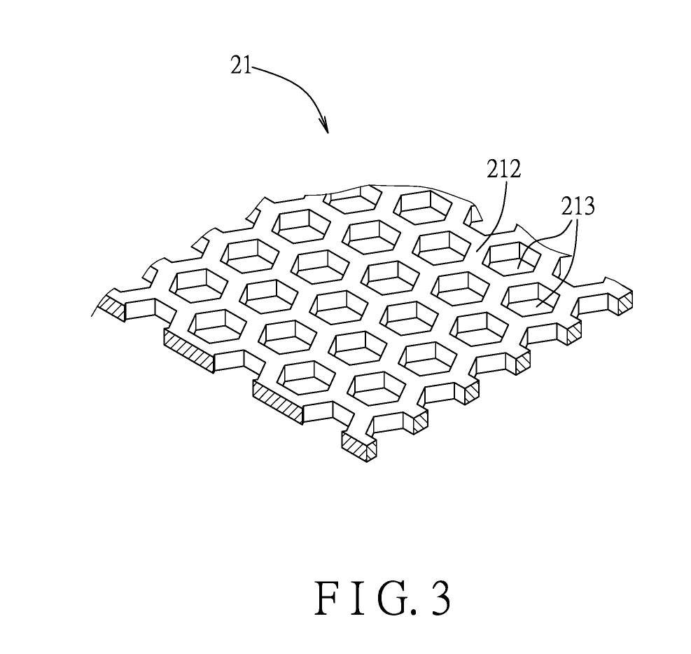

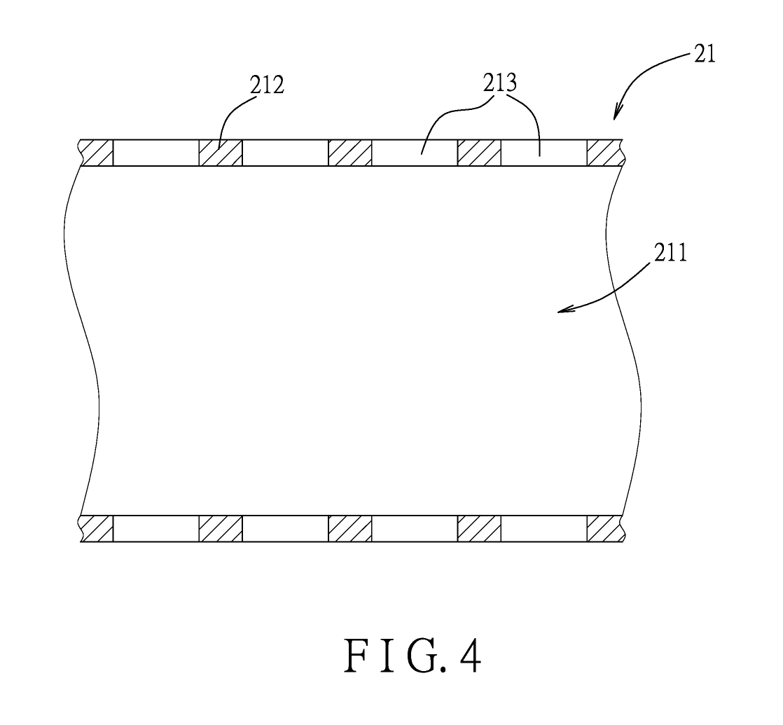

Referring further to FIGS. 3 and 4, Step S1 of the method in this embodiment is to provide the screen mold 21. The screen mold 21 is made of a non-pliable material, and has an annular surrounding wall 212 defining an internal space 211, and a plurality of screen holes 213 extending through the annular surrounding wall 212 and communicating spatially with the internal space 211. In this embodiment, the screen mold 21 is connected to a mold-operating device 3 for driving the screen mold 21 to move linearly and to rotate. The mold-operating device 3 includes a linear-moving member 32 and a rotating member 31 that is connected to the linear-moving member 32 and the screen mold 21. The mold-operating device 3 can be actuated so as to linearly move the screen mold 21 through the linear-moving member 32 and/or to rotate the screen mold 21 about a rotation axis through the rotating member 31. In this embodiment, the linear-moving member 32 is able to move in at least six linear directions, including leftward, rightward, upward, downward, forward and rearward directions, so that the screen mold 21 can be moved in these directions.

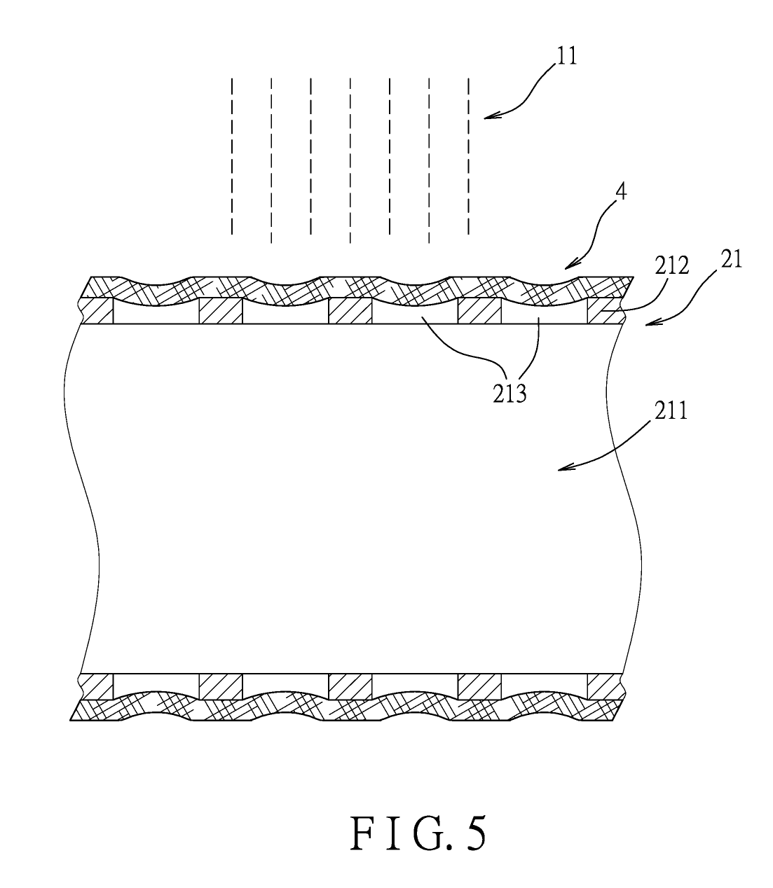

Referring to FIGS. 1 and 5, Step S2 of the method in this embodiment is disposing a formable material over the annular surrounding wall 212 and the screen holes 213 of the screen mold 21. The formable material may be a melt-blown fabric material such as the molten fibers 11. More specifically, in Step S2 of this embodiment, the molten fibers 11 produced by the melt-blown device 1 are deposited on an outer surface of the screen mold 21. In this embodiment, a polymeric raw material is used to form the molten fibers 11 using the melt-blown device 1. The thus-formed molten fibers 11 are then sprayed onto the screen mold 21, and the molten fibers 11 are draped over the screen mold 21 to form a melt-blown fiber layer 4 that covers the annular surrounding wall 212 and the screen holes 213 of the screen mold 21. Referring to FIG. 2, during Step S2, the mold-operating device 3 can be actuated so as to linearly move the screen mold 21 through the linear-moving member 32 and/or to rotate the screen mold 21 about a rotation axis through the rotating member 31 for controlling coverage of the molten fibers 11 on the screen mold 21. Since the mold-operating device 3 can be driven to rotate and move the screen mold 21 in various directions, the locations on the outer surface of the screen mold 21 where the melt-blown fiber layer 4 is to be formed can be selected according to different needs.

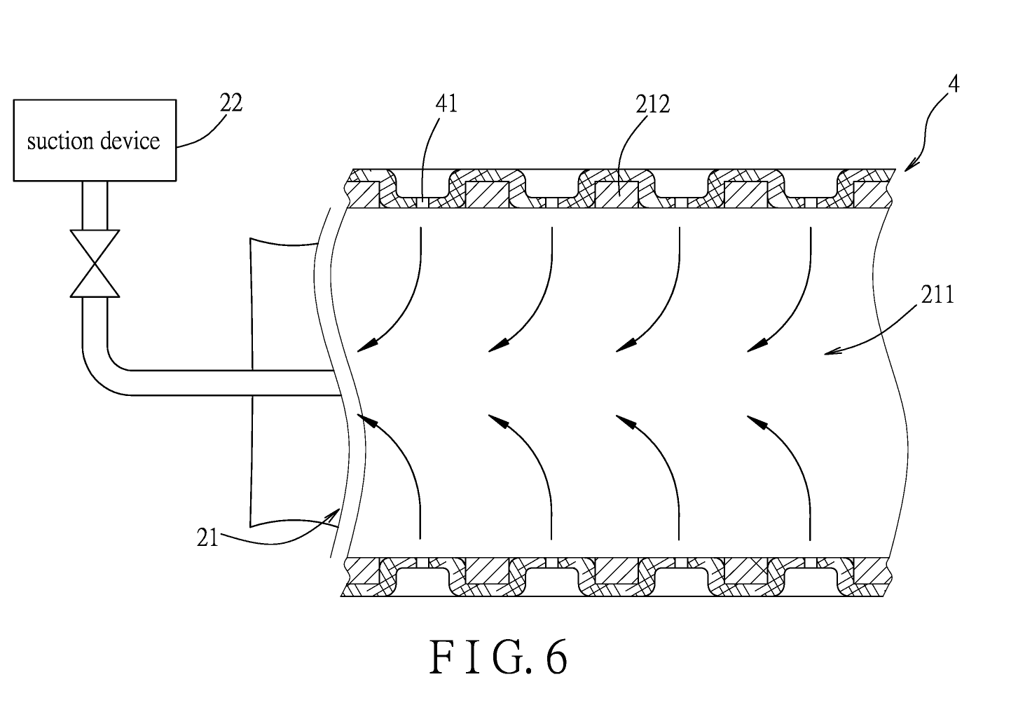

Referring to FIGS. 1 and 6, Step S3 of the method in this embodiment is to evacuate air from the internal space 211 of the screen mold 21 via the suction device 22 of the apparatus 2 to form the formable material into the fabric article. In this embodiment, the formable material is a melt-blown fabric material, namely, the molten fibers 11. The melt-blown fiber layer 4 formed from the molten fibers 11 in Step S2 retains a certain degree of plasticity when not completely cured. When the air in the internal space 211 is evacuated by the suction device 22, the ambient pressure outside the internal space 211 is greater than the internal pressure of the internal space 211. Thus, the melt-blown fiber layer 4 with plasticity is pressed against the screen mold 21, and a plurality of breathable holes 41 that correspond in position to the screen holes 213 are formed. The melt-blown fiber layer 4 is then cured into the non-woven fabric article. It is worth mentioning that the breathable holes 41 are capable of enhancing the breathability and the easy-drying effect of the non-woven fabric article without undermining the original merits of the non-woven fabric article. In this embodiment, the diameter of the later-formed breathable holes 41 is much larger than that of the original pores of the melt-blown fiber layer 4.

Referring back to FIG. 2, in this embodiment, the screen mold 21 of the apparatus 2 generally has a shape of an upper human body part (which includes the chest, abdomen, upper arm portions, and optionally the neck), and the non-woven fabric article formed in Step S3 is a breathable garment having such shape. In this embodiment, specifically, the annular surrounding wall 212 of the screen mold 21 is generally shaped into the aforesaid upper human body part. The screen mold 21 has two first regions 214 that correspond in position to axillary regions of the aforesaid upper human body part, and a second region 215 that interconnects the first regions 214 and that corresponds in position to the remaining regions of the aforesaid upper human body part excluding the axillary regions. The screen holes 213 in the first regions 214 are distributed more densely than those in the second region 215.

Since the screen mold 21 in this embodiment generally has a shape of the aforesaid upper human body part, the cured melt-blown fiber layer 4 can be formed integrally into a fabric article that consists of a one-piece non-woven wearable unit having two seamless non-woven sleeves, thereby dispensing with the time and cost for sewing multiple non-woven fabric articles into a single garment. In addition, since the axillary regions of the upper human body part are more prone to perspiration, the screen holes 213 of the screen mold 21 are distributed more densely at the first regions 214 (which correspond to the axillary regions) than at the second region 215 (which corresponds to the remaining regions of the upper human body part). Thus, the axillary regions of the non-woven fabric article molded by the screen mold 21 have good air permeability. However, the present disclosure is not limited to this feature, as the number and density of the screen holes 213 can be adjusted according to actual needs. Moreover, the diameter of the screen holes 213 can be adjusted to produce non-woven fabric articles having different sizes of the breathable holes 41.

It should be particularly noted that, since the formable material of this embodiment is the melt-blown fiber layer 4 (i.e., the molten fibers 11), the process of evacuating air from the screen mold 21 to form the breathable holes 41 has to be carried out before the melt-blown fiber layer 4 is cured, so that a breathable non-woven fabric article can be formed after the curing of the melt-blown fiber layer 4. In other words, the resulting air-permeable non-woven fabric article has a plurality of the breathable holes 41. In addition, in order to directly form a breathable non-woven garment, the screen mold 21 may be shaped into a human body part. However, in other embodiments, the formable material may be a plastic film or a composite cloth attached with a plastic film (not shown in the drawings), and thus, the shape of the screen mold 21 is not limited to the shape of a human body part as the plastic film or the composite cloth can be of any arbitrary shape. In certain embodiments, the plastic film or the composite cloth can be directly sleeved on the screen mold 21 and thereafter subjected to suction to form a breathable plastic film or a breathable composite cloth having a plurality of the breathable holes 41. Therefore, Step S2 (disposing the molten fibers 11 over the screen mold 21 to form to the melt-blown fiber layer 4) is omitted. It is worth mentioning that, if the formable material is a plastic film or a composite cloth, the screen mold 21 will be attached to a heat source (not shown in the drawings) in order to heat the plastic film or the composite cloth, resulting in a semi-molten plastic film or composite cloth. Therefore, the plasticity of the plastic film or the composite cloth can be enhanced with increased adhesion to the screen mold 21, and air is thereafter evacuated from the screen mold 21 to form a non-woven fabric pattern having a plurality of the breathable holes 41. As for the above-mentioned composite cloth, any one of a woven fabric, a non-woven fabric and a garment can be selected to be attached to a plastic film to form such composite cloth, according to the user's needs.

In summary, the method for making a non-woven fabric article according to the present disclosure includes evacuating air using a suction device 22 to form, in the melt-blown fiber layer 4, a plurality of holes that correspond in position to the screen holes 213. Following the curing of the melt-blown fiber layer 4, a non-woven fabric article having a plurality of the breathable holes 41 is formed. The breathability and the easy-drying effect of the non-woven fabric article of this disclosure can be enhanced by the breathable holes 41, and the original merits of the non-woven fabric article can be maintained.

In the description above, for the purposes of explanation, numerous specific details have been set forth in order to provide a thorough understanding of the embodiment. It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects.

While the disclosure has been described in connection with what is considered the exemplary embodiment, it is understood that this disclosure is not limited to the disclosed embodiment but is intended to cover various arrangements included within the spirit and scope of the broadest interpretation so as to encompass all such modifications and equivalent arrangements.

What is claimed is:

1. A method of making a non-woven fabric article, comprising:
(a) providing a screen mold made of a non-pliable material, and having an annular surrounding wall that defines an internal space, and a plurality of screen holes that extend through the annular surrounding wall and spatially communicate with the internal space;
(b) disposing a formable material over the annular surrounding wall and the screen holes of the screen mold; and
(c) evacuating air from the internal space via a suction device to form the formable material into the fabric article.

2. The method as claimed in claim 1, wherein:
in step (b), the formable material is a melt-blown fabric material; and
in step (c), the melt-blown fabric material is formed into the fabric article.

3. The method as claimed in claim 2, wherein the screen mold has a shape of a human body part, and the fabric article is a breathable garment.

4. The method as claimed in claim 3, wherein the human body part is an upper human body part, and the screen holes of the screen mold which correspond in position to axillary regions of the human body part are distributed more densely than the screen holes of the screen mold which correspond in position to the remaining regions of the human body part.

5. The method as claimed in claim 1, further comprising: connecting the screen mold to a mold-operating device that includes a linear-moving member and a rotating member; and actuating the mold-operating device during the disposition of the formable material so as to linearly move the screen mold through the linear-moving member and to rotate the screen mold about a rotation axis through the rotating member.

6. The method as claimed in claim 1, further comprising: connecting the screen mold to a mold-operating device that includes a linear-moving member and a rotating member; and actuating the mold-operating device during the disposition of the formable material so as to linearly move the screen mold through the linear-moving member.

7. The method as claimed in claim 1, further comprising: connecting the screen mold to a mold-operating device that includes a linear-moving member and a rotating member; and actuating the mold-operating device during the disposition of the formable material so as to rotate the screen mold about a rotation axis through the rotating member.

8. The method as claimed in claim 5, wherein the linear-moving member is operable to move leftward, rightward, upward, downward, forward and rearward so that the screen mold is movable leftward, rightward, upward, downward, forward and rearward.

9. An apparatus for making a non-woven fabric article, comprising:
a screen mold made of a non-pliable material, and having an annular surrounding wall that defines an internal space, and a plurality of screen holes that extend through said annular surrounding wall and spatially communicate with said internal space; and
a suction device connected to said screen mold and operable to evacuate air from said internal space.

10. The apparatus as claimed in claim 9, wherein said screen mold has a first region and a second region connected to said first region, said screen holes in said first region being distributed more densely than those in said second region.

11. The apparatus as claimed in claim 9, wherein said screen mold has a shape of a human body part, said annular surrounding wall being shaped into an upper portion of the human body part.

12. The apparatus as claimed in claim 11, wherein said screen mold has a first region and a second region connected to said first region, said screen holes in said first region being distributed more densely than those in said second region, said first region corresponding in position to an axillary region of the upper portion of the human body part.

13. A fabric article comprising a non-woven wearable unit having a seamless non-woven sleeve.
